(19) United States
(12) Patent Application Publication
(10) Pub. No.: US 2024/0312630 A1
(43) Pub. Date: Sep. 19, 2024

(54) DYNAMIC SENSING AND INTERVENTION SYSTEM

(71) Applicant: Zimmer US, Inc., Warsaw, IN (US)

(72) Inventors: Dave Van Andel, Spring Lake, MI (US); Mark Brincat, Moreton in Marsh (GB); Ted Spooner, Grand Rapids, MI (US)

(21) Appl. No.: 18/576,700

(22) PCT Filed: Jul. 15, 2022
(86) PCT No.: PCT/US2022/037284

§ 371 Date: Jan. 4, 2024

Related U.S. Application Data
(60) Provisional application No. 63/222,665, filed on Jul. 16, 2021.

(51) Int. Cl. G16H 50/20; G06F 21/62; G16H 20/40; G16H 40/63; G16H 40/67

(52) U.S. Cl. CPC G16H 50/20; G06F 21/6245; G16H 20/40; G16H 40/63; G16H 40/67

(57) ABSTRACT

Systems and techniques may be used to determine what device to use to process data in an implanted sensor data processing system. An example technique may include determining, based on patient-specific information, whether to use a local machine learning model operable at a mobile device, or a remote machine learning model operable at a remote device to output a prediction generated using sensor data. The example technique may include, in accordance with a determination that the local machine learning model is to be used, predicting, at the mobile device, an outcome for the patient using the local machine learning model. The example technique may include, in accordance with a determination that the remote machine learning model is to be used, sending, from the mobile device, the compiled data to a remote computing device to generate a predicted outcome.

### CLAIM OF PRIORITY

This application claims the benefit of priority to U.S. Provisional Application No. 63/222,665 filed Jul. 16, 2021, titled “DYNAMIC SENSING AND INTERVENTION SYSTEM,” which is hereby incorporated herein by reference in its entirety.

### BACKGROUND

Orthopedic patient care may require surgical intervention, such as for lower extremities (a knee, a hip, etc.). For example, when pain becomes unbearable for a patient, surgery may be recommended. Postoperative care may include immobility of a joint ranging from weeks to months, physical therapy, or occupational therapy. Physical therapy or occupational therapy may be used to help the patient with recovering strength, everyday functioning, and healing. Current techniques involving immobility, physical therapy, or occupational therapy may not monitor or adequately assess range of motion or for pain before or after surgical intervention.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates example implantable devices with embedded sensors in accordance with at least one example of this disclosure.

FIG. 2 illustrates an implanted sensor data processing system in accordance with at least one example of this disclosure.

FIG. 3 illustrates a communication diagram for an implanted sensor data processing system in accordance with at least one example of this disclosure.

FIG. 4 illustrates a machine learning engine for determining feedback in accordance with at least one example of this disclosure.

FIG. 5 illustrates a flowchart showing a technique for determining what device to use to process data in an implanted sensor data processing system in accordance with at least one example of this disclosure.

FIG. 6 illustrates a flowchart showing a technique for mapping sensor data to wearable data in accordance with at least one example of this disclosure.

FIG. 7 illustrates a flowchart showing a technique for providing feedback regarding a patient-specific goal related to recovery from an orthopedic procedure in accordance with at least one example of this disclosure.

FIG. 8 illustrates a block diagram of an example machine upon which any one or more of the techniques discussed herein may perform in accordance with at least one example of this disclosure.

### DETAILED DESCRIPTION

Systems and techniques described herein may be used for providing postoperative feedback to a patient or provider. The postoperative feedback may include feedback related to recovery from an orthopedic procedure. For example, the feedback may include information related to pain management, physical therapy, range of motion, movement speed or acceleration, stiffness, likelihood of a need for further surgical or medical intervention, or the like.

Systems and techniques described herein may be used to monitor patient progress, provide updates, and modify a postoperative recovery plan. A model may be trained (e.g., using machine learning techniques as described herein) to predict outcomes related to the patient. For example, a mobile device may receive compiled data generated by a sensor embedded in an orthopedic implant in a patient, label the compiled data with wearable device generated data, and send the compiled data to a remote computing device, such as a server, a cloud computing device, a desktop computer, etc. The remote computing device may generate a model using machine learning and send the model to the mobile device. The mobile device may use the model (which may be personalized to the patient) to output a prediction.

In some examples, separate (but optionally related) models may be stored at a mobile device and a remote computing device. The mobile device may determine, for example based on patient-specific information, whether to use a local machine learning model stored on the mobile device or a remote machine learning model stored on the remote computing device to output a prediction. In accordance with a determination that the local machine learning model is to be used, an outcome may be predicted for the patient by using compiled data as an input to the local machine learning model. In accordance with a determination that the remote machine learning model is to be used, compiled data may be sent to the remote computing device to predict an outcome using the remote machine learning model. The models may differ in complexity, such that the remote model may be more accurate, but the local model may be quicker or require less processing. The determination of which model to use may be based on how accurate a prediction is needed, when a prediction was last obtained, a nature of urgency, pain, or risk to the patient, or the like.

In an example, a model may be selected based on patient data. A plurality of machine learning models may be trained using implanted sensor data from corresponding patient populations. Embedded sensor data may be used to select one of the plurality of machine learning models, optionally using additional information specific to the patient. The selected machine learning model may be used to output a prediction for the patient.

An embedded device embedded in an orthopedic implant in a patient may include memory, communication circuitry, a sensor to generate data, or processing circuitry (which may include an integrated circuit, such as a system on a chip, a field-programmable gate array (FPGA), a processor, etc.). The embedded device may be used to generate, store, or send data.

Using data generated by the embedded device, wearable device generated data may be validated, which may improve predicted outcomes. For example, a time series of sensor data may be generated by a sensor embedded in an orthopedic implant in a patient, and a time series of wearable data may be generated by a wearable device worn by the patient. A model may be generated that maps the time series of sensor data to the time series of wearable data. The model may be used to approximate or estimate sensor data using only wearable data. This may limit the need for sensor data while maintaining accuracy. From time to time, the model may be updated or revalidated using sensor data and wearable data.

In an example, a model generated or using embedded sensor data may be used to detect and predict outcomes related to patient-specific goals (e.g., “when can I golf,” “when can I go up stairs,” “when can I reach up to the cupboard,” “when can I play with my grandkids,” etc.). The patient-specific goal related to recovery from an orthopedic procedure on a patient may be converted into a set of one or more metrics. The sensor data and the model may be used to determine whether metrics of the set of one or more metrics are satisfied. In response to determining that all metrics of the set of one or more metrics are satisfied, an indication that the patient-specific goal has been achieved may be output (e.g., a message on a user interface, audio, or the like, such as indicating “congratulations, you have met your goal and now may do activity X”). In response to determining that a metric of the set of one or more metrics is not satisfied, an indication corresponding to the metric may be output (e.g., encouragement or details about how to achieve the goal, additional education information, or the like).

In some examples, an embedded sensor device may be pre-configured, such as with modes that are already validated (e.g., from a regulatory standpoint). These modes may be activated when needed, and due to the pre-configuration and validation, may not need to be subject to additional regulatory review. For example, an implantable sensor device configured to be embedded in an orthopedic implant in a patient may include memory with a table having information identifying a plurality of applications, the plurality of applications preconfigured to be validated under a regulatory system and an indication of whether each application is currently active. The implantable sensor device may include communication circuitry to receive an indication to activate one of the plurality of applications. In response, the identified application may be activated (e.g., by changing its status in the table). In an example, at least one operation of a sensor of the implantable sensor device is changed when the one of the plurality of applications is initiated or activated.

These systems and techniques may improve patient outcomes. Using an implanted sensor, intelligent data may be collected with a tiered architecture for processing the data. The systems and techniques described herein may be used to validate and improve accuracy of data sources, activate real-time follow-up, configure event-based changes to data collection frequency and fidelity (e.g., in response to a detected drop in walking speed), or the like. In some examples, an application (e.g., an app) may be used, such as on a mobile device to push a request to the sensor to increase frequency of data collection or transmission. In an example, the sensor may be dormant, and activated based on need (e.g., using information generated by a gyroscope or by activating a gyroscope).

In some examples, battery life may be considered or sacrificed based on current needs. A finite battery life of an embedded sensor may be managed. For example, the sensor device may be placed into a sleep or semi-sleep mode, where the sensor device checks periodically for data or periodically outputs data (e.g., every hour, day, 15 minutes, etc.). The length of time between periods may be adjusted in accordance with patient need and battery life management.

In an example, data may be collected locally at a mobile device (e.g., a smart phone), such as to build a patient profile (e.g., a patient-specific model). The patient profile may be used to output predictive information. When the implanted sensor communicates with the mobile device, the mobile device may send a message to the implanted sensor to change modes (e.g., wake up into a more intense mode, download a software or firmware update, etc.).

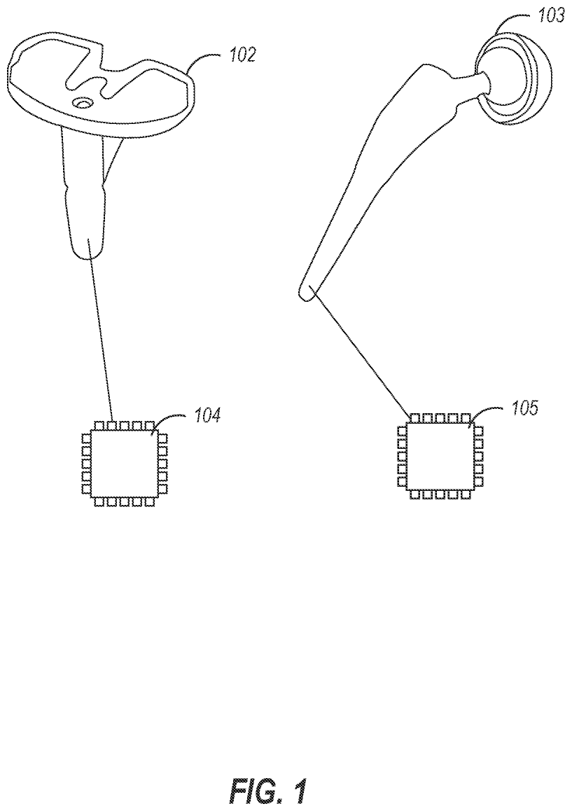

FIG. 1 illustrates example implantable devices 102 and 103 with embedded sensors 104 and 105 in accordance with at least one example of this disclosure. The embedded sensors 104 and 105 may be the same, or may be configured differently for a particular implant. Implantable device 102 is an example tibial implant (e.g., at a proximal tibia) and implantable device 103 is an example hip implant (e.g., at a proximal femur). The embedded sensors 104 and 105 may be part of a device (e.g., an embedded sensor device) that is configured to be inserted in a stem portion of one of the implantable devices 102 or 103. In some examples, the embedded sensor device may be configured to be inserted into either of the implantable devices 102 or 103, or may be specific to the implant (e.g., configured for the particular stem, such as a particular arrangement of a housing or components of the embedded sensor device). The implantable device 102 may be implanted as part of a knee replacement procedure (e.g., a partial or total knee arthroplasty) and the implantable device 103 may be implanted as part of a hip replacement procedure. Other orthopedic procedures that use an implant may be used with an embedded sensor, such as a shoulder replacement procedure, a spine procedure (e.g., in a pedicle screw), or the like.

In an example, the embedded sensors 104 and 105 may be validated before being inserted into the implantable devices 102 and 103, respectively. Validation for the embedded sensors 104 and 105 may include submission to a regulatory body (e.g., of a country, state, etc.), and receipt of validation from the regulatory body. A device containing one of the embedded sensors 104 and 105 may be validated together or separately from applications or uses of the embedded sensors 104 and 105. In an example, the embedded sensors 104 and 105 may include one or more applications that are validated but not activated when inserted into the implantable devices 102 or 103, when the implantable devices 102 or 103 are implanted into a patient, or during a surgical procedure. In this example, the one or more validated application may be in an inactive state, and may be later activated (e.g., postoperatively), such as when needed for data collection, data compiling, data storage, or the like. In an example, multiple sensors may be present in a sensor device, and one or more of the multiple sensors may initially be inactive. Applications or sensors may be activated by a remote device (e.g., a mobile device, such as a phone) using an app or otherwise communicating with the sensor device housing the applications or sensors. Applications that are pre-validated may include circuitry (e.g., a processor, software, firmware, hardwired circuitry, etc.) that is capable of performing various functions, which are initially disabled, and later enabled when needed.

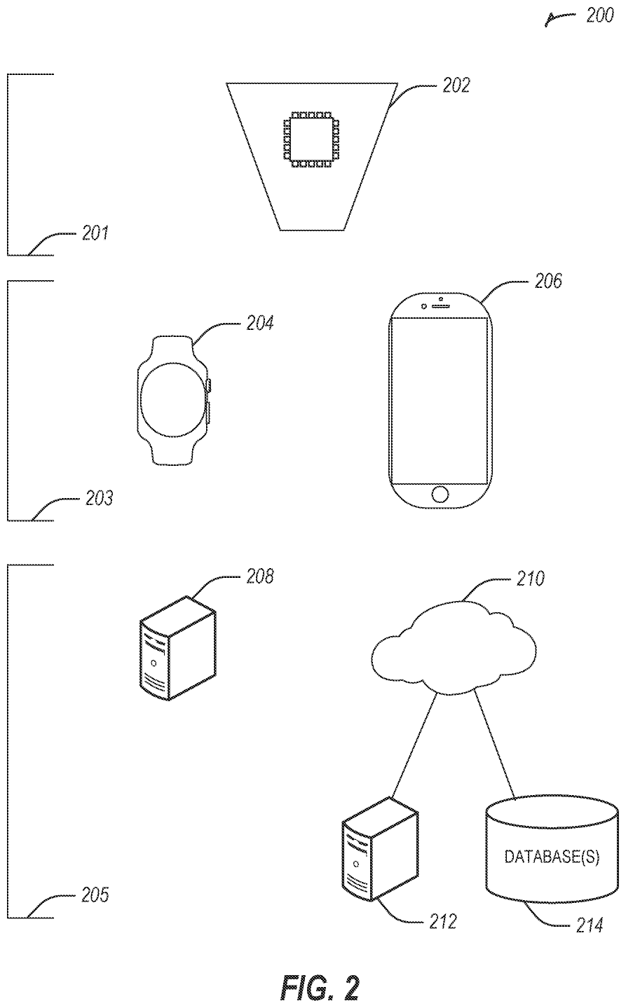

FIG. 2 illustrates an implanted sensor data processing system 200 in accordance with at least one example of this disclosure. The processing system 200 may operate with one or more levels having different processing power, processing capabilities, battery availability, heat requirements, costs, time limits, or the like. A first level 201 may correspond to processing done at an implantable sensor device 202 (e.g., one that is implanted into a patient). Processing done at the first level 201 may include limited data gathering (e.g., receiving sensor data, storing sensor data, etc.), some compiling, such as adding a timestamp to sensor data, or compiling sensor data into a single file for transmission, or the like. In some examples, processing at the first level 201 may include determining a battery status, memory status, or the like. In an example, processing at the first level 201 may be dependent on timing, such as length of time since a surgical procedure. For example, in an initial period (e.g., a day, a week, a month, six months), more processing may be done (e.g., more frequent sampling of sensor data, more frequent transmission of sensor data to a remote device, etc.). Then, in a subsequent period, less processing may be done (e.g., less frequent sampling or transmission of sensor data). In an example, sensor data received at the implantable sensor device 202 may be checked for anomalies (e.g., whether the data is within a particular range), such as for temperature, acceleration, etc.

A second level 203 includes devices that may be in proximity to the implantable sensor device 202, such as devices in communication (e.g., direct communication) with the implantable sensor device 202 (e.g., via Bluetooth, Wi-Fi, Wi-Fi direct, via an RFID or other NFC technology, etc.). Devices at the second level 203 may include those with more processing power, capabilities, memory, batteries, or the like than the implantable sensor device 202 of the first level 201, but may still have limitations. Example devices of the second level 203 include a wearable device 204 or other internet of things device, a mobile phone 206, a tablet, or the like. Some of these devices may have different limitations than others (e.g., processing power of the mobile device 206 may be greater than that of the wearable device 204).

A third level 205 includes devices with the greatest processing power, capabilities, power, etc. of system 200. Devices in the third level 205 may include a computer 208 (e.g., desktop or laptop), cloud-based devices 210 (e.g., a server 212), which may include access to a database 214, or the like.

Each of the three levels of devices may be used for different types or degrees of processing. For example, the first level 201 may be used for local sensor computing, the second level 203 may be used for middle mobile device computing (e.g., implementing or updating a machine learning model), and the third level 205 may be used for remote server computing (e.g., generating or updating a machine learning model). The tiered architecture of the system 200 may take advantage of remote and local processing capabilities.

In an example, the first level 201 may be used for local processing, such as edge computing on the sensor device. This may include using a basic analytics model uploaded to the implantable sensor device 202. In an example, the implantable sensor device 202 may identify anomalous events independent of mobile device or server interaction. In some examples, the implantable sensor device 202 may enter into an “emergency mode” in real-time (e.g., to shut down due to heat or battery usage, to increase sensor data capture due to identifying a potential issue, or the like).

The second level 203 may be used for to run a more sophisticated analytics model, for example at the mobile device 206. The model may use sensor data from the implantable sensor device 202. In some examples, the model may use additional inputs, such as PROMs, data from the wearable device 204, video recordings of activities, inputs from a patient (e.g., a pain rating, a comfort rating, etc.), range of motion information, or the like. The model may provide interventions in near real time.

The third level 205 may provide server-based processing of a larger population data sets. For example, the mobile device 206 may send data to the cloud 210 (e.g., compiled data that is stripped of personally identifiable information), which may be used with data from other patients. The server 212 may develop a model to be pushed out to the mobile device 206 or the implantable sensor device 202. Different models may be developed (e.g., for different patient populations, for different devices such as the mobile device 206 and the implantable sensor device 202, for different patient timelines such as during a first six months and after six months, or the like). For example, in a first six months or twelve months after a surgical procedure, more rigorous data collection may occur (e.g., every hour), then after the initial period, the implantable sensor device 202 may transition into a hibernation or check-in mode, which may include obtaining data on demand (e.g., from a signal from the mobile device 206 or the wearable device 204), once a day, once a week, or the like.

The various levels may be used at different time intervals. For example, the ultra-localized processing in the implantable sensor device 202 may be performed multiple times a day, patient-specific monitoring may be performed at the mobile device 206 (e.g., daily), and population analytics may be performed at the server 212, such as weekly or monthly. In an example, ultra-localized processing includes cleaning data, generating electrical signals from raw data, processing the data to send summary data to the mobile device 206, optionally some refined outputs, for example not just raw data but compiled data, or the like. The data may be collected at the implantable sensor device 202 for example three times a day, every 5-6 hours, after a first movement read, etc. The data may be sent at each interval to the mobile device 206 or may be held until a sufficient amount of data is stored and sent together.

In an example, anomalies may be alerted using a model at the mobile device 026, the wearable device 204, or the server 212. In response to detecting an anomaly, a change to operation of the implantable sensor device 202 may be sent from the alerting device. The implantable sensor device 202 may change data collection methods, for example changing to a more intense mode (e.g., every fifteen minutes, every hour, etc.), such as for infection detection, loosening of the implant, etc. In some examples, even when no modeling is done at the second level 203, the devices of the second level 203 may receive data from the implantable sensor device 202 to remove personal data before sending to devices of the third level 205.

In an example, a model may be generated for use with data from the implantable sensor device 202. However, as discussed above, the implantable sensor device 202 has limited resources, such as battery power (which may not be rechargeable in some examples). Data from the wearable device 204 may be readily available and not have battery issues. However, the data from the wearable device 204 may be less accurate than the data from the implantable sensor device 202. In a technique, over time, wearable data may be used as a proxy for implantable sensor data. In this technique, a first model is used for alerting, predicting, or monitoring a patient using the implantable sensor data. A second model or classifier may be used to map wearable data to the implantable sensor data. For example, the wearable data may be labeled with the implantable sensor data or with events (e.g., predictions, alerts, etc.). In one version of the technique, the second model may be used to directly translate wearable data into synthetic implantable sensor data. For example, the wearable data may be input into the second model, and synthetic implantable sensor data may be output from the second model. This synthetic implantable sensor data may then be input into the first model, as if it had been generated by the implantable sensor device 202. In a second version of the technique, the second model may be trained to directly use the wearable data to output alerts, predictions, or the like.

This technique may be used to validate and improve accuracy of data sources, such as the wearable data described above. In some examples, instead of or in addition to the wearable data, mobile device data may be used. The technique may be used to improve accuracy of alerts or predictions for data regarding step counts, for example. In this example, captured gait characteristics of the patient (e.g., from the implantable sensor data) may be used to adjust the model of gait stored by measuring devices and used to estimate step count or other metrics.

Alert detections and predictions may be used to a detect or predict abnormal patient state or a change in patient state requiring intervention based on significant difference from population norm, for example. In some examples, the technique described above may be used to calibrate the wearable device 204 or the mobile device 206. After the second model or classifier is trained or generated, the alerts or predictions may occur without implantable sensor data (e.g., without activating or receiving data from the implantable sensor device 202).

In an example, the wearable data may be used to predict when more frequent implantable sensor data may be useful or needed. For example, the wearable data may monitor the patient to predict how the patient is likely to progress, using the second model and activating the implantable sensor device 202.

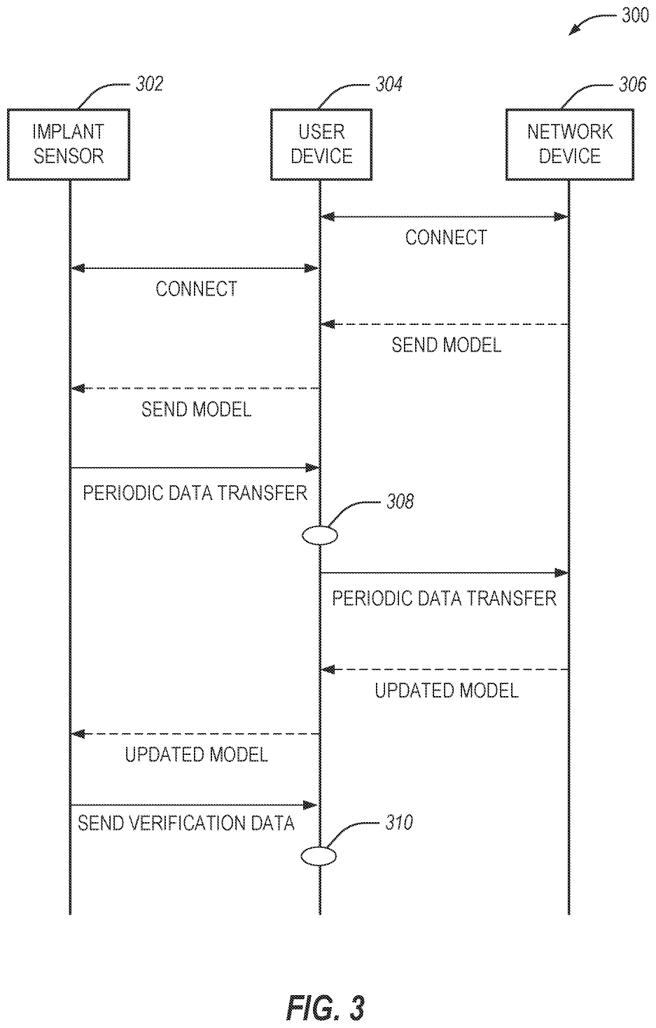

FIG. 3 illustrates a communication diagram 300 for an implanted sensor data processing system in accordance with at least one example of this disclosure. The diagram 300 shows communication between an implantable sensor 302 (e.g., that is within an implant in a patient), a user device 304 (e.g., a wearable device, a mobile device such as a phone, a computer, etc.), and a network dev ice 306 (e.g., a server, a cloud device, etc.).

The communication diagram 300 illustrates a connection step between the implantable sensor 302 and the user device 304 and a connection step between the network device 306 and the user device 304. These connection steps may occur in any order, may be initiated by any of the devices, and may occur before or after a surgical procedure to implant the implantable sensor 302.

In an example, a model may be pre-loaded on the user device 304 or the implantable sensor 302. In this example, an update to the model may be sent from the network device 306 to the user device 304 (for the user device 304, for the implantable sensor 302, or for both) or from the user device 304 to the implantable sensor 302. In another example, a model may be sent from the network device 308 to the user device 304 for loading on the user device 304, the implantable sensor 302, or both. The user device 304 may send the model to the implantable sensor 302.

The implantable sensor 302 may periodically transfer data to the user device 304. After one or more periodic data transfers, the user device 304 may perform analysis on the data at time 308. The analysis may include removing personally identifiable information from the data, running the data through a model to determine whether to output an alert or a prediction, or compiling the data for sending to the network device 306. The user device 304 may periodically transfer data (e.g., forward the same data sent by the implantable sensor 302, or send data in a different format based on the data sent by the implantable sensor 302, such as data with personal information removed, compiled data, model results, etc.) to the network device 306. Using information, such as the data sent by the user device 304, data generated by other implantable sensors, wearable sensor data, data collected at the user device 304, or the like, the network device 306 may generate an updated model or an update to a model. The updated model or update to the model may be sent to the user device 304, and then optionally to the implantable sensor 302 from the user device 304. In some examples, the implantable sensor 302 may send verification data to the user device 304. The verification data may be used to verify wearable data generated at a wearable device (not shown). The wearable data may be generated as described above with respect to FIG. 2.

A model may use data from the implantable sensor 302 to provide an alert or prediction related to patient outcomes, procedures, or movement. For example, the model may relate to gait monitoring, range of motion monitoring, physical therapy compliance monitoring, other clinical evaluations (e.g., risk of manipulation under anesthesia, risk of infection, risk of deterioration, risk of revision, risk of pain, etc.), or the like.

Predictive analytics may be used to drive a change in sensor configuration dynamically. For example, an output of a model run on the network device 306, the user device 304, or the implantable sensor 302 may be used to change a parameter of the implantable sensor 302, such as increasing frequency of data collection, changing type of data collection, or changing how data is compiled or stored. For example, a model may monitor for a high risk of need for manipulation under anesthesia, infection detection, or the like. The implantable sensor 302 may change operation from a normal or standard mode to a “manipulation under anesthesia” mode or an “infection detection” mode, for example.

An XML-based configuration file may be used to change settings of the implantable sensor 302. An intelligent data collection system with a tiered architecture for adapting sensor configurations to environmental or patient data changes may be used.

In an example, a model may be used for predictive analytics. The model may generate predictive details for a patient based on data from the implantable sensor 302, such as what the patient is likely to complete, such as from a recovery program. Patient pathways are multi-layer and a route through clinical progression includes treatment and recovery, medications, or include self-management may be specific to the patient. Using the implantable sensor 302 data, the patient may optimize their own individual recovery.

Predictive analytics may be used to predict patient-specific outcomes by considering whether the patient is active (e.g., resulting in a higher likelihood the patient will need more surgical intervention), or whether the patient is likely to over-exercise and damage the body or cause harm during recovery. The implantable sensor 302 data may be used for refinements during recovery, such as to create or modify a personalized recovery programs, based on patient data, a model, or data or model generated from other patients' data (e.g., without personal information). External sensor data (e.g., wearable sensor data) may not be accurate enough to evaluate these types of results and make these types of predictions (e.g., wearable data may only be available to around 5 degrees in range of motion). Some data may be available from the implantable sensor 302 that is not available in a wearable, such as vibrations, load (e.g., force or pressure), internal temperature, etc. The implantable sensor 302 may include a force sensor, a pressure sensor, an accelerometer, a gyroscope, an inertial measurement unit (IMU), a pedometer, a thermometer, a conductance sensor, a thermal sensor, a vibration sensor, or the like.

Predictions may be based on personal goals or targets, and a prediction may be output to a user with a personalized message. For example, a user may set a goal of walking up stairs, playing golf, or sitting on the floor. The model may generate a prediction based on range of motion, mobility, data from the implantable sensor 302, etc. This prediction may be used to generate a personalized user message, such as “you're not ready for a full flight of stairs yet, but try two stairs.” The personalized user message may be based on a classification of the patient goal into movement or load needs. A plan may be generated to help the patient achieve the goal, and data from the implantable sensor 302 may be used to predict when the goal will be achieved, how the goal may be achieved, and to inform the patient what to expect throughout the journey. In an example, real world environmental personal details (e.g., floor plan of house, number of stairs in house, daily walking area, environmental considerations such as hills, city or rural, weather, kids or grandkids, etc.) may be used with the model to generate a prediction. In an example, an initial prediction may be sent to a care team with later predictions sent to a patient. In an example, a prediction may be used as a warning, for example if movement indicative of stair use is detected and the prediction indicates that the patient is not ready for stairs, a message may be sent to the patient (e.g., via user device 304) warning the patient to not attempt stairs yet.

In an example, the implantable sensor 302 may be preloaded with a plurality of applications (e.g., configurations, uses, programs, circuitry such as a printed circuit board (PCB), system on a chip (SoC), field-programmable gate array (FPGA), or other integrated circuit or hardware level types of applications, etc.) that are initially not activated. The applications may be validated (e.g., by a regulatory body) before installation in an implant or a patient. The functions of the applications may be validated before installation, but not activated until after installation in an implant or a patient. An application of the not activated applications may be remotely activated (e.g., using xml or other software instructions, such as in a message from the user device 304). The implantable sensor 302 may be operably modified to a different programmability capability (e.g., to change the data collection, output, model, etc.), for example changing what the implantable sensor 302 does or changing data collection frequency.

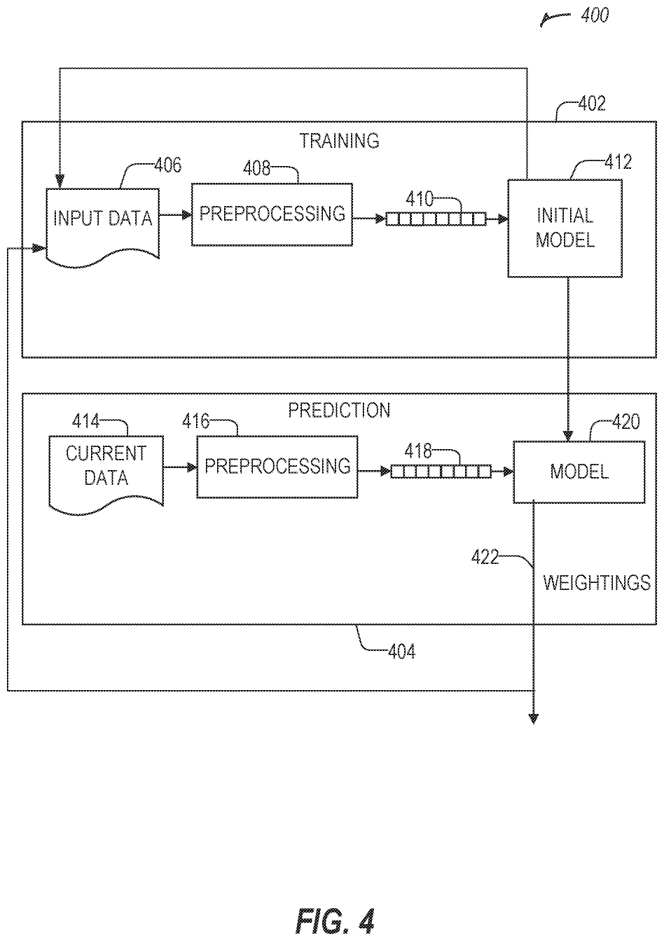

FIG. 4 illustrates a machine learning engine for determining feedback in accordance with some embodiments. The machine learning engine can be employed within a mobile device (e.g., a cell phone) or a computer. A system may calculate one or more weightings for criteria based upon one or more machine learning algorithms. FIG. 4 shows an example machine learning engine 400 according to some examples of the present disclosure.

Machine learning engine 400 utilizes a training engine 402 and a prediction engine 404. Training engine 402 uses input data 406, after undergoing preprocessing 408, to determine one or more features 410. The one or more features 410 may be used to generate an initial model 412, which may be updated iteratively or with future unlabeled data.

The input data 406 may be labeled with an indication, such as a degree of success of an outcome of a surgical procedure or patient recovery, such as pain information, patient feedback, implant success, ambulatory information, range of motion, specific goals (e.g., movement goals, exercise goals, action goals such as lifting items, playing a sport, or driving a car, or the like). In some examples, an outcome may be subjectively assigned to input data, but in other examples, one or more labelling criteria may be utilized that focuses on outcome metrics (e.g., range of motion, pain rating, survey score, a patient satisfaction score, such as a forgotten knee score, a WOMAC score, shoulder assessment, hip assessment, or the like).

In the prediction engine 404, current data 414 may be input to preprocessing 416. In some examples, preprocessing 416 and preprocessing 408 are the same. The prediction engine 404 produces feature vector 418 from the preprocessed current data, which is input into the model 420 to generate one or more criteria weightings 422. The criteria weightings 422 may be used to output a prediction, as discussed further below.

The training engine 402 may operate in an offline manner to train the model 420 (e.g., on a server). The prediction engine 404 may be designed to operate in an online manner (e.g., in real-time, at a mobile device, on an implant device, etc.). In other examples, the training engine 402 may operate in an online manner (e.g., at a mobile device). In some examples, the model 420 may be periodically updated via additional training (e.g., via updated input data 406 or based on unlabeled data output in the weightings 422) or user feedback (e.g., an update to a technique or procedure). The initial model 412 may be updated using further input data 406 until a satisfactory model 420 is generated. The model 420 generation may be stopped according to user input (e.g., after sufficient input data is used, such as 1,000, 10,000, 100,000 data points, etc.) or when data converges (e.g., similar inputs produce similar outputs).

The specific machine learning algorithm used for the training engine 402 may be selected from among many different potential supervised or unsupervised machine learning algorithms. Examples of supervised learning algorithms include artificial neural networks, Bayesian networks, instance-based learning, support vector machines, decision trees (e.g., Iterative Dichotomiser 3, C4.5, Classification and Regression Tree (CART), Chi-squared Automatic Interaction Detector (CHAID), and the like), random forests, linear classifiers, quadratic classifiers, k-nearest neighbor, linear regression, logistic regression, and hidden Markov models. Examples of unsupervised learning algorithms include expectation-maximization algorithms, vector quantization, and information bottleneck method. Unsupervised models may not have a training engine 402. In an example embodiment, a regression model is used and the model 420 is a vector of coefficients corresponding to a learned importance for each of the features in the vector of features 410, 418.

Data input sources for the model 420 may include one or more of an implanted sensor device, a watch, a fitness tracker, a wrist-worn device, a sweat monitor (e.g., electrolytes level), a blood-sugar monitor (e.g., for diabetes), a heart monitor (e.g., EKG or ECG), a heart rate monitor, a pulse oximeter, a stress level monitor (e.g., via an Apple watch), a respiratory rate monitor device, a ‘life-alert,’ an ear wearable (e.g., for measuring intercranial pressure, such as via the tympanic membrane), a head attached wearable, an ultrasound wearable, a microphone dysphonia device, a smart contact, a smart ring, exercise equipment (e.g., elliptical, mirror, treadmill, bike like peloton, stair stepper, etc.), a phone app that tracks data, an intraoperative data collection device (e.g., vision and robotic information), a chest band (e.g., for respiration and heart rate), video, or the like.

Input data for the model 420 may include user input information, app data, such as from a food app, an exercise tracker, etc., a response to a questionnaire/PROM, video capture (e.g., for range of motion or strength), pain levels, opioid usage, compliance (e.g., with PT or OT or education steps), education data, exercise data, demographic or family history information, cognitive tests, BMI, exercise (daily/weekly/monthly), work status (unemployed, working, retired), age, gender, income/wealth status, children, marital status, or the like. Other input information to the model 420 may include clinician side data, a patient profile (e.g., demographics, preferences, etc.), a medical history of the patient, imaging, an arthritis lab panel, or the like.

Once trained, the model 420 may output a correlated sensor data-based outcome from an input of non-implanted sensor data. In this example, the input data 406 may include sensor data, labeled with wearable data. Each type of data may be saved as a time-series to correlate the sensor data and the wearable data. A separate model may be generated to indicate outcomes for sensor data, for example based on sensor data labeled with outcomes (e.g., patient predictions, evaluations, etc.). The model 420 may generate a set of features that correlate the sensor data to the wearable data such that the wearable data may be used to approximate the sensor data. The output of model 420 may include a mapping or feature set to convert wearable data to sensor data such that wearable data may be input to the model 420, which may generate sensor data, which may be used in the separate model to provide an outcome. In this manner, only wearable data may be used to generate outcomes, without the need for or use of sensor data after training.

In another example, the model 420 may predict a patient readiness score or indication related to whether a patient is ready to perform a particular action, exercise, or specific goal. In an example, this model 420 may be used as the above separate model. This model 420 may be generated using sensor data, wearable data, or any of the other data described above. The output of this model 420 may include identifying whether or when a patient may be able to achieve a specific goal, such as a movement goal, an exercise goal, lifting an item, playing a sport, driving a car, or the like.

In an example, the model 420 may be used to predict whether a patient is a high-risk patient. When a patient is identified as high risk using the model 402, an implanted sensor device may be operated at a higher throughput of obtaining and outputting sensor data. The model 420 may predict high-risk for a patient when there is a higher risk of infection, higher risk of need for manipulation under anesthesia, higher risk of pain, etc.

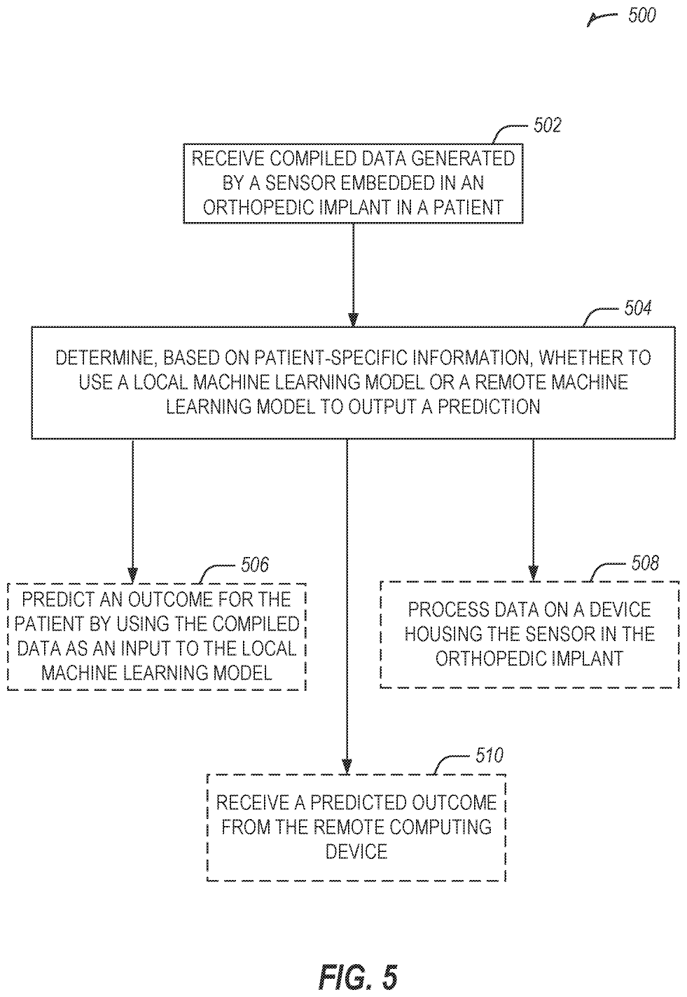

FIG. 5 illustrates a flowchart showing a technique 500 for determining what device to use to process data in an implanted sensor data processing system in accordance with at least one example of this disclosure. The technique 500 may be performed by processing circuitry of a device, such as an implanted sensor device, a wearable device, a mobile device (e.g., a phone, a tablet, etc.), a computer (e.g., a laptop, a desktop, a server, etc.), or the like, locally, on an edge device, or in the cloud.

The technique 500 includes an operation 502 to receive compiled data generated by a sensor embedded in an orthopedic implant in a patient. The compiled data may include data pre-processed by the sensor device.

The technique 500 includes an operation 504 to determine, based on patient-specific information, whether to use a local machine learning model or a remote machine learning model to output a prediction. In an example, the local machine learning model is run on a mobile device and the remote machine learning model is run on a remote server. In response to the determination at operation 504, various operations may be selected as a next step. For example, when the processing may be completed locally, the technique 500 proceeds with operation 506. When a more accurate, more difficult, or more extensive determination needs to be made, the technique 500 may proceed with operation 510. When a simple operation (e.g., aggregation) is to be done, the technique 500 may proceed to operation 508, in an example.

Operation 504 may include a determination related to an orthopedic procedure performed on the patient. For example, the determination may be based on a current time frame, such as when the procedure occurred within the time frame (e.g., in the last six months), more extensive determinations may be made, for example using operation 510. In this example, operation 510 may be used according to a schedule, such as once a day or once a week, while operations 506 and 508 occur more frequently than after the time period. In an example, operation 510 may be not used after a time period (e.g., after twelve months after the procedure). In other examples, the type of procedure, location of the procedure, complications from the procedure, or the like may be used in the determination.

Operation 504 may use patient specific information to determine a next step. For example, a pain level identified by the patient, a range of motion of the patient, a patient exercise score, patient progress towards a goal defined or selected by the patient, or the like may be used to determine whether to collect more or less data (e.g., operation 508), use operation 506 or 510 (or both), or the like. In an example, when the patient is experiencing more pain than an average patient or baseline, additional information may be obtained by the sensor, for example at operation 508. In this example, the remote operation 510 may be used to obtain more accurate information. In another example, when the patient has a risk of complications, the local device operation 506 may be used for faster and more secure results.

The technique 500 includes an optional operation 506 to predict an outcome for the patient by using the compiled data as an input to the local machine learning model. The technique 500 includes an optional operation 508 to process data on a device housing the sensor in the orthopedic implant. The technique 500 includes an optional operation 510 to receive a predicted outcome from the remote computing device. Operation 510 may occur in response to sending the compiled data to the remote computing device. In an example, before the complied data is sent to the remote computing device, the compiled data may be sanitized to remove personally identifying information from the compiled data. In some examples, a prediction may be generated in the least amount of time on the device housing the sensor. In some examples, a prediction may be generated in less time using the local machine learning model (e.g., on a mobile device or nearby device to the sensor) than the time taken by the remote computing device. In some examples, accuracy of predictions may be inversely proportional to time taken to generate the predictions.

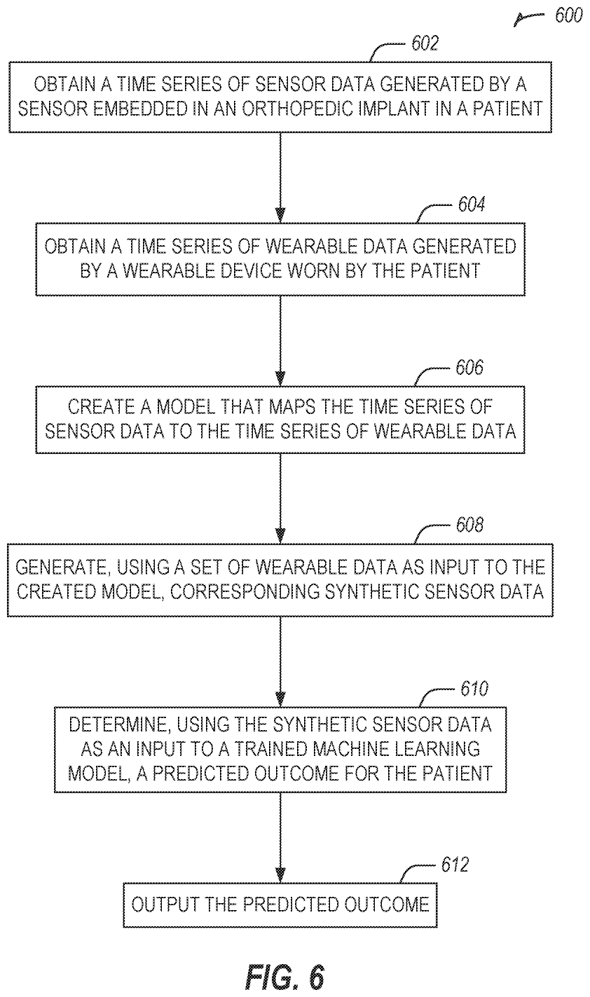

FIG. 6 illustrates a flowchart showing a technique for mapping sensor data to wearable data in accordance with at least one example of this disclosure.

The technique 600 includes an operation 602 to obtain a time series of sensor data generated by a sensor embedded in an orthopedic implant in a patient. The technique 600 includes an operation 604 to obtain a time series of wearable data generated by a wearable device worn by the patient. In an example, the time series of sensor data or the time series of wearable data may include at least one of accelerometer, gyroscope, force data, gait data, or the like. The time series of data may be generated from a smartwatch.

The technique 600 includes an operation 606 to create a model that maps the time series of sensor data to the time series of wearable data. The model may map the time series of sensor data and the time series of wearable data based on timestamps of respective data points of each time series occurring within a threshold time range of each other. For example, data points within a few milliseconds, a few seconds, etc., may be correlated.

The technique 600 includes an operation 608 to generate, using a set of wearable data as input to the created model, corresponding synthetic sensor data. The mapping may be used to mimic real sensor data using the wearable data.

The technique 600 includes an operation 610 to determine, using the synthetic sensor data as an input to a trained machine learning model, a predicted outcome for the patient. In some examples, the trained machine learning model is trained to output at least one of patient progress towards a rehabilitation goal or a remaining life span of the orthopedic implant. The trained machine learning model may be trained using the times series of sensor data or other sensor data. Operation 610 may include determining the predicted outcome without using any generated sensor data (e.g., only using the synthetic sensor data or the wearable data). Synthetic data may include generated data, representative data, or the like. In an example, operation 610 may include using fewer input data points generated by the sensor than would be needed to output a prediction without the synthetic sensor data. For example, if several hundred generated sensor data points are needed to accurately output a prediction, the synthetic data may be used with only a few tens or hundreds of generated sensor data to accurately output a prediction.

The technique 600 includes an operation 612 to output the predicted outcome. The prediction may be output for display on a user interface, for example (e.g., to a clinician, a patient, etc.).

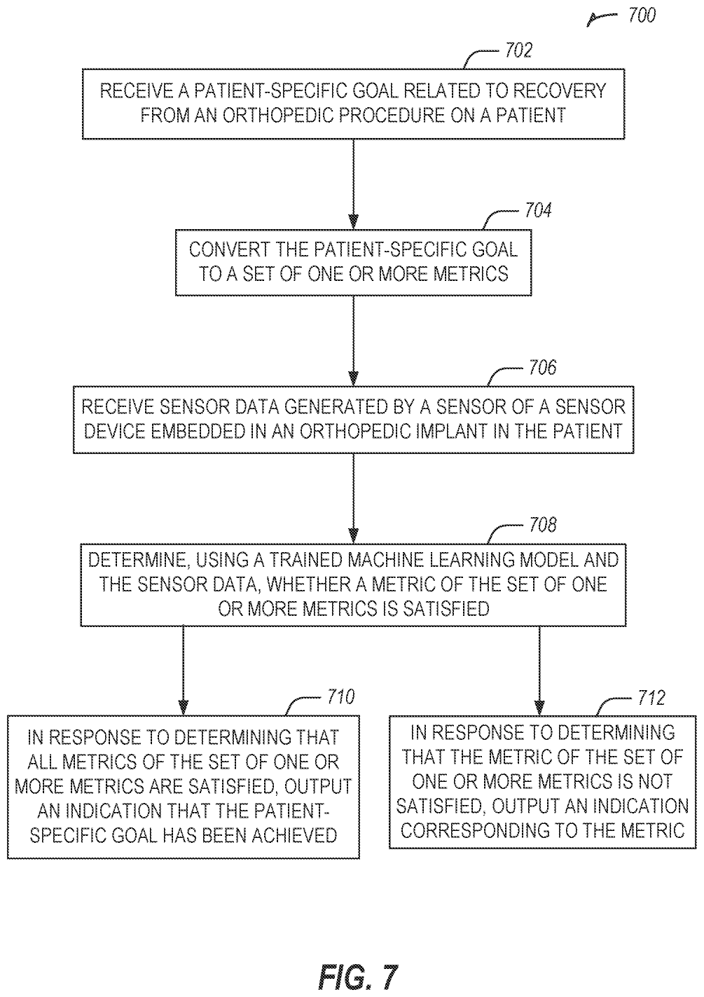

FIG. 7 illustrates a flowchart showing a technique for providing feedback regarding a patient-specific goal related to recovery from an orthopedic procedure in accordance with at least one example of this disclosure.

The technique 700 includes an operation 702 to receive a patient-specific goal related to recovery from an orthopedic procedure on a patient. The technique 700 includes an operation 704 to convert the patient-specific goal to a set of one or more metrics. The one or more metrics may include a range of motion for a joint. The patient-specific goal may include an identification of a life activity. In this example, the set of one or more metrics may include a range of motion corresponding to the life activity. The patient-specific goal may be received via a selection by the patient of the patient-specific goal on a user interface.

The technique 700 includes an operation 706 to receive sensor data generated by a sensor of a sensor device embedded in an orthopedic implant in the patient. The technique 700 includes a decision operation 708 to determine, using a trained machine learning model and the sensor data, whether a metric of the set of one or more metrics is satisfied. The trained machine learning model may be trained using historical sensor data with metric completion labels.

The technique 700 includes an operation 710 to, in response to determining at operation 708 that all metrics of the set of one or more metrics are satisfied, output an indication that the patient-specific goal has been achieved. The indication that the patient-specific goal has been achieved may include information corresponding to a second patient-specific goal. For example, goals may be set up sequentially or a stretch goal may be available (e.g., “I want to golf nine holes” may become “I want to golf 18 holes”).

The technique 700 includes an operation 712 to, in response to determining at operation 708 that the metric of the set of one or more metrics is not satisfied, output an indication corresponding to the metric. The indication corresponding to the metric may include an identified exercise to improve the metric (e.g., physical therapy, stretches, walking, etc.). In some examples, the indication may include a predicted date when the patient-specific goal will be achieved.

The technique 700 may include sending, to the sensor device, an indication to activate one of a plurality of applications stored in memory of the sensor device, the plurality of applications preconfigured to be validated under a regulatory system. The received sensor data may be generated according to the activated application. In some examples, at least one operation of the sensor may be changed when the activated application is activated.

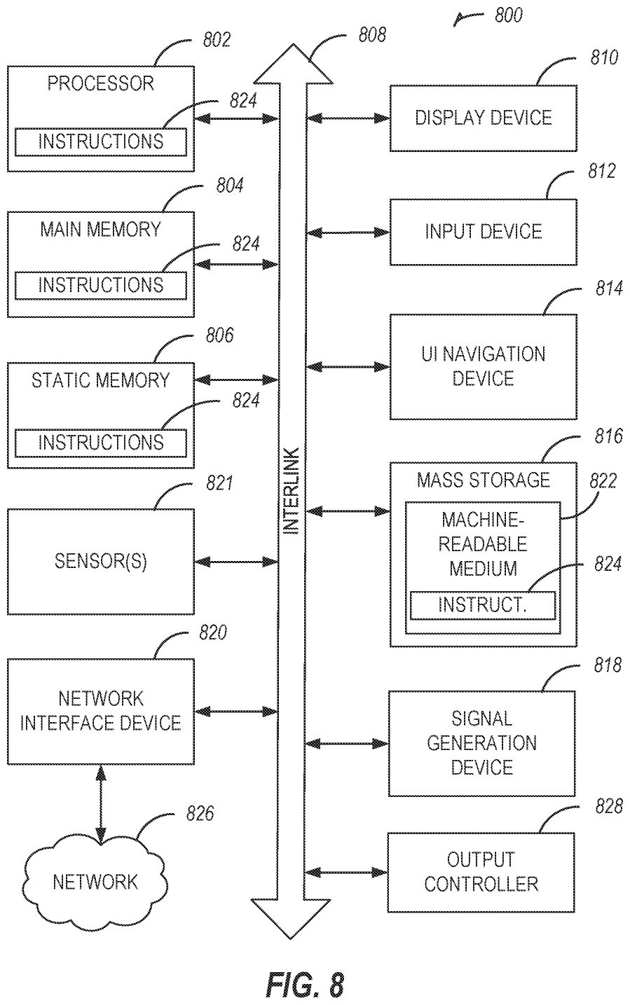

FIG. 8 illustrates a block diagram of an example machine 800 upon which any one or more of the techniques discussed herein may perform in accordance with some embodiments. This example machine can operate some or all of the Orthopedic Intelligence System discussed herein. In some examples, the Orthopedic Intelligence System can operate on the example machine 800. In other examples, the example machine 800 is merely one of many such machines utilized to operate the Orthopedic Intelligence System. In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 800 may include a hardware processor 802 (e.g., a central processing unit (CPU), a graphics processing unit (GPU), a hardware processor core, or any combination thereof), a main memory 804 and a static memory 806, some or all of which may communicate with each other via an interlink (e.g., bus) 808. The machine 800 may further include a display unit 810, an alphanumeric input device 812 (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 816, a signal generation device 818 (e.g., a speaker), a network interface device 820, and one or more sensors 821, such as a global positioning system (GPS) sensor, compass, accelerometer, or other sensor. The machine 800 may include an output controller 828, such as a serial (e.g., Universal Serial Bus (USB), parallel, or other wired or wireless (e.g., infrared (IR), near field communication (NFC), etc.) connection to communicate or control one or more peripheral devices (e.g., a printer, card reader, etc.).

The storage device 816 may include a machine readable medium 822 on which is stored one or more sets of data structures or instructions 824 (e.g., software) embodying or utilized by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 816 may constitute machine readable media.

While the machine readable medium 822 is illustrated as a single medium, the term “machine readable medium” may include a single medium or multiple media (e.g., a centralized or distributed database, and/or associated caches and servers) configured to store the one or more instructions 824. The term “machine readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine-readable medium examples may include solid-state memories, and optical and magnetic media.

The instructions 824 may further be transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software.

Each of the following non-limiting examples may stand on its own, or may be combined in various permutations or combinations with one or more of the other examples.

Example 1 is a method comprising: receiving, at a mobile device, compiled data generated by a sensor embedded in an orthopedic implant in a patient; labeling the compiled data with at least one of data generated by the mobile device, data generated by a wearable communicatively coupled to the mobile device, or data input by a user at the mobile device; sending the compiled data to a remote computing device; receiving, from the remote computing device, a machine learning model; receiving, at the mobile device, a second set of compiled data generated by the sensor; determining, using the second set of compiled data as an input to the machine learning model, a prediction; and outputting the prediction.

Example 2 is a method comprising: receiving, at a server from a mobile device, a first set of compiled data generated by a sensor embedded in an orthopedic implant in a patient and labeled at the mobile device; generating, at the server, a machine learning model using the first set of compiled data; sending the machine learning model to the mobile device; receiving, at the server from the mobile device, a second set of compiled data generated by the sensor; updating the machine learning model at the server based on the second set of compiled data; and maintaining a copy of the machine learning model and the updated machine learning model at the server.

Example 3 is a method comprising: receiving, at a mobile device, compiled data generated by a sensor embedded in an orthopedic implant in a patient; determining, based on patient-specific information, whether to use a local machine learning model or a remote machine learning model to output a prediction; in accordance with a determination that the local machine learning model is to be used, predict an outcome for the patient by using the compiled data as an input to the local machine learning model; in accordance with a determination that the remote machine learning model is to be used: sending the compiled data to a remote computing device; and receiving a predicted outcome from the remote computing device.

In Example 4, the subject matter of Example 3 includes, wherein determining whether to use the local machine learning model or the remote machine learning model includes determining a current time frame related to an orthopedic procedure performed on the patient.

In Example 5, the subject matter of Examples 3-4 includes, wherein determining whether to use the local machine learning model or the remote machine learning model is based on an orthopedic procedure previously done on the patient.

In Example 6, the subject matter of Examples 3-5 includes, wherein determining whether to use the local machine learning model or the remote machine learning model includes using a pain level identified by the patient, a range of motion of the patient, or a patient exercise score.

In Example 7, the subject matter of Examples 3-6 includes, wherein determining whether to use the local machine learning model or the remote machine learning model includes determining patient progress towards a goal defined by the patient.

In Example 8, the subject matter of Examples 3-7 includes, wherein sending the compiled data includes sanitizing the compiled data before sending to remove personally identifying information from the complied data.

Example 9 is a method comprising: receiving, at a mobile device, a plurality of machine learning models from a remote computing device, the plurality of machine learning model trained using implanted sensor data from corresponding patient populations; receiving, at the mobile device, a first set of compiled data generated by a sensor embedded in an orthopedic implant in a patient; selecting one of the plurality of machine learning models based on the first set of compiled data and information specific to the patient; receiving a second set of compiled data generated by the sensor; and determining, using the second set of compiled data as an input to the one of the plurality of machine learning models, a predicted outcome for the patient.

Example 10 is a device embedded in an orthopedic implant in a patient, the device comprising: memory; communication circuitry; a sensor to generate data; and processing circuitry to: determine whether to locally process the generated data or send the generated data to a device outside the patient; in accordance with a determination to locally process the generated data, aggregate the generated data and store the generated data in the memory; and in accordance with a determination to send the generated data to the device outside the patient: activate the communication circuitry to send the generated data to the device outside the patient; and receive, from the device outside the patient based on processing done on the device outside the patient, an updated instruction for the sensor.

In Example 11, the subject matter of Example 10 includes, wherein the device outside the patient is a base station, a mobile device, a wearable device, or a computer.

In Example 12, the subject matter of Examples 10-11 includes, wherein the device outside the patient is to remove personally identifying information before sending data to a further device.

In Example 13, the subject matter of Examples 10-12 includes, wherein the updated instruction for the sensor includes increasing a rate of sensor data collection, based on a determination that a risk of need for manipulation under anesthesia or infection is above a threshold.

In Example 14, the subject matter of Examples 10-13 includes, wherein the communication circuitry is to send the generated data as a time series of data usable at the device outside the patient to validate measurements of a wearable device.

In Example 15, the subject matter of Examples 10-14 includes, wherein the generated data is usable by the device outside the patient to provide an alert to the patient advising the patient of progress towards a goal.

Example 16 is a method comprising: obtaining, at a processor, a time series of sensor data generated by a sensor embedded in an orthopedic implant in a patient; obtaining, at the processor, a time series of wearable data generated by a wearable device worn by the patient; generating a model that maps the time series of sensor data to the time series of wearable data; determining, using a set of wearable data as input to the model, corresponding synthetic sensor data; and determining, using the synthetic sensor data as an input to a machine learning model, a predicted outcome for the patient.

Example 17 is at least one machine-readable medium including instructions, which when executed by processing circuitry, cause the processing circuitry to perform operations to: receive a patient-specific goal related to recovery from an orthopedic procedure on a patient; convert the patient-specific goal to a set of one or more metrics; receive sensor data generated by a sensor embedded in an orthopedic implant in the patient; determine, using a machine learning model and the sensor data, whether metrics of the set of one or more metrics are satisfied; in response to determining that all metrics of the set of one or more metrics are satisfied, output an indication that the patient-specific goal has been achieved; and in response to determining that a metric of the set of one or more metrics is not satisfied, output an indication corresponding to the metric.

In Example 18, the subject matter of Example 17 includes, wherein the indication corresponding to the metric includes an identified exercise to improve the metric.

In Example 19, the subject matter of Examples 17-18 includes, wherein the indication corresponding to the metric includes a predicted date when the patient-specific goal will be achieved.

In Example 20, the subject matter of Examples 17-19 includes, wherein the one or more metrics includes a range of motion for a joint.

Example 21 is an implantable sensor device configured to be embedded in an orthopedic implant in a patient comprising: a sensor; processing circuitry; and memory, the memory including: table including: a plurality of applications, the plurality of applications preconfigured to be validated under a regulatory system; and an activation status corresponding to each of the plurality of applications; communication circuitry to receive an indication to activate one of the plurality of applications; wherein the table is updated to indicate the one of the plurality of applications is active in response to receiving the indication; and wherein at least one operation of the sensor is changed when the processing circuitry initiates the one of the plurality of applications.

Example 22 is a mobile device comprising: processing circuitry; and memory including instructions, which when executed by the processing circuitry, cause the processing circuitry to perform operations to: receive, at a mobile device, compiled data generated by a sensor of a sensor device embedded in an orthopedic implant in a patient; determine, based on patient-specific information, whether to use a local machine learning model operable at the mobile device, or a remote machine learning model operable at a remote device to output a prediction generated using the compiled data; in accordance with a determination that the local machine learning model is to be used, predict, at the mobile device, an outcome for the patient by using the compiled data as an input to the local machine learning model; in accordance with a determination that the remote machine learning model is to be used, send, from the mobile device, the compiled data to a remote computing device to generate a predicted outcome at the remote computing device using the compiled data.

In Example 23, the subject matter of Example 22 includes, wherein the compiled data includes data pre-processed by the sensor device.

In Example 24, the subject matter of Examples 22-23 includes, wherein to determine whether to use the local machine learning model or the remote machine learning model includes operations to determine a current time frame related to an orthopedic procedure performed on the patient, and comparing the current time frame to a threshold time frame.

In Example 25, the subject matter of Examples 22-24 includes, wherein to determine whether to use the local machine learning model or the remote machine learning model includes operations to determine which model to use based on identifying an orthopedic procedure previously done on the patient.

In Example 26, the subject matter of Examples 22-25 includes, wherein to determine whether to use the local machine learning model or the remote machine learning model includes operations to determine which model to use based on an input received at the mobile device of at least one of a pain level identified by the patient, a range of motion of the patient, or a patient exercise score.

In Example 27, the subject matter of Examples 22-26 includes, wherein to determine whether to use the local machine learning model or the remote machine learning model includes operations to determine patient progress towards a goal defined by the patient.

In Example 28, the subject matter of Examples 22-27 includes, wherein to send the compiled data includes operations to sanitize the compiled data before being sent to remove personally identifying information from the compiled data.

In Example 29, the subject matter of Examples 22-28 includes, wherein a prediction by the local machine learning model is obtained in less time than a prediction by the remote machine learning model.

Example 30 is at least one machine-readable medium, including instructions for operation at a mobile device, which when executed, cause processing circuitry to perform operations to: receive, at a mobile device, compiled data generated by a sensor of a sensor device embedded in an orthopedic implant in a patient; determine, based on patient-specific information, whether to use a local machine learning model operable at the mobile device, or a remote machine learning model operable at a remote device to output a prediction generated using the compiled data; in accordance with a determination that the local machine learning model is to be used, predict, at the mobile device, an outcome for the patient by using the compiled data as an input to the local machine learning model; in accordance with a determination that the remote machine learning model is to be used, send, from the mobile device, the compiled data to a remote computing device to generate a predicted outcome at the remote computing device using the compiled data.

In Example 31, the subject matter of Example 30 includes, wherein the compiled data includes data pre-processed by the sensor device.

In Example 32, the subject matter of Examples 30-31 includes, wherein to determine whether to use the local machine learning model or the remote machine learning model includes operations to determine a current time frame related to an orthopedic procedure performed on the patient, and compare the current time frame to a threshold time frame.

In Example 33, the subject matter of Examples 30-32 includes, wherein to determine whether to use the local machine learning model or the remote machine learning model includes operations to determine which model to use based on identifying an orthopedic procedure previously done on the patient.

In Example 34, the subject matter of Examples 30-33 includes, wherein to determine whether to use the local machine learning model or the remote machine learning model includes operations to determine which model to use based on an input received at the mobile device of at least one of a pain level identified by the patient, a range of motion of the patient, or a patient exercise score.

In Example 35, the subject matter of Examples 30-34 includes, wherein to determine whether to use the local machine learning model or the remote machine learning model includes operations to determine patient progress towards a goal defined by the patient.

In Example 36, the subject matter of Examples 30-35 includes, wherein to send the compiled data includes operations to sanitize the compiled data before being sent to remove personally identifying information from the compiled data.

In Example 37, the subject matter of Examples 30-36 includes, wherein a prediction by the local machine learning model is obtained in less time than a prediction by the remote machine learning model.

Example 38 is a system comprising: a sensor device embedded in an orthopedic implant in a patient, the sensor device including: a sensor to generate data; and processing circuitry to compile the data; and a mobile device including: communication circuitry; processing circuitry; and memory including instructions, which when executed by the processing circuitry, cause the processing circuitry to perform operations to: determine, based on patient-specific information, whether to use a local machine learning model operable at the mobile device, or a remote machine learning model operable at a remote device to output a prediction generated using the compiled data; in accordance with a determination that the local machine learning model is to be used, predict, at the mobile device, an outcome for the patient by using the compiled data as an input to the local machine learning model; in accordance with a determination that the remote machine learning model is to be used, send, from the communication circuitry of the mobile device, the compiled data to a remote computing device to generate a predicted outcome at the remote computing device using the compiled data.

In Example 39, the subject matter of Example 38 includes, wherein the compiled data includes data pre-processed by the processing circuitry within the sensor device.

In Example 40, the subject matter of Examples 38-39 includes, wherein to determine whether to use the local machine learning model or the remote machine learning model includes operations to use at least one of a current time frame related to an orthopedic procedure performed on the patient, identification of an orthopedic procedure previously done on the patient, or an input received at the mobile device including at least one of a pain level identified by the patient, a range of motion of the patient, or a patient exercise score.

In Example 41, the subject matter of Examples 38-40 includes, wherein to send the compiled data includes operations to sanitize the compiled data before being sent to remove personally identifying information from the compiled data.

Example 42 is a method comprising: obtaining, at a processor, a time series of sensor data generated by a sensor embedded in an orthopedic implant in a patient; obtaining, at the processor, a time series of wearable data generated by a wearable device worn by the patient; creating a model that maps the time series of sensor data to the time series of wearable data; generating, using a set of wearable data as input to the created model, corresponding synthetic sensor data, determining, using the synthetic sensor data as an input to a trained machine learning model, a predicted outcome for the patient; and outputting the predicted outcome.

In Example 43, the subject matter of Example 42 includes, wherein the trained machine learning model is trained to output at least one of patient progress towards a rehabilitation goal or a remaining life span of the orthopedic implant.

In Example 44, the subject matter of Examples 42-43 includes, wherein the trained machine learning model is trained using the times series of sensor data.

In Example 45, the subject matter of Examples 42-44 includes, wherein the model maps the time series of sensor data and the time series of wearable data based on timestamps of respective data points of each time series occurring within a threshold time range of each other.

In Example 46, the subject matter of Examples 42-45 includes, wherein determining the predicted outcome for the patient occurs without using any generated sensor data as an input.

In Example 47, the subject matter of Examples 42-46 includes, wherein determining the predicted outcome for the patient occurs using fewer input data points generated by the sensor than would be needed to output a prediction without the synthetic sensor data.

In Example 48, the subject matter of Examples 42-47 includes, wherein the time series of sensor data includes at least one of accelerometer or gyroscope data, and wherein the time series of wearable data includes gait data.

In Example 49, the subject matter of Examples 42-48 includes, wherein obtaining the time series of data generated by the wearable device includes receiving the time series of data from a smartwatch.

Example 50 is a device comprising: processing circuitry; and memory including instructions, which when executed by the processing circuitry, cause the processing circuitry to perform operations to: obtain a time series of sensor data generated by a sensor embedded in an orthopedic implant in a patient; obtain a time series of wearable data generated by a wearable device worn by the patient; create a model that maps the time series of sensor data to the time series of wearable data; generate, using a set of wearable data as input to the created model, corresponding synthetic sensor data; and determine, using the synthetic sensor data as an input to a trained machine learning model, a predicted outcome for the patient; and output the predicted outcome.

In Example 51, the subject matter of Example 50 includes, wherein the trained machine learning model is trained to output at least one of patient progress towards a rehabilitation goal or a remaining life span of the orthopedic implant.

In Example 52, the subject matter of Examples 50-51 includes, wherein the trained machine learning model is trained using the times series of sensor data.

In Example 53, the subject matter of Examples 50-52 includes, wherein the model maps the time series of sensor data and the time series of wearable data based on timestamps of respective data points of each time series occurring within a threshold time range of each other.

In Example 54, the subject matter of Examples 50-53 includes, wherein the predicted outcome for the patient is determined without using any generated sensor data as an input.

In Example 55, the subject matter of Examples 50-54 includes, wherein the predicted outcome for the patient is determined using fewer input data points generated by the sensor than would be needed to output a prediction without the synthetic sensor data.

In Example 56, the subject matter of Examples 50-55 includes, wherein the time series of sensor data includes at least one of accelerometer or gyroscope data, and wherein the time series of wearable data includes gait data.

In Example 57, the subject matter of Examples 50-56 includes, wherein the wearable device is a smartwatch.

Example 58 is at least one machine-readable medium, including instructions, which when executed, cause processing circuitry to perform operations to: obtain a time series of sensor data generated by a sensor embedded in an orthopedic implant in a patient; obtain a time series of wearable data generated by a wearable device worn by the patient; create a model that maps the time series of sensor data to the time series of wearable data; generate, using a set of wearable data as input to the created model, corresponding synthetic sensor data; and determine, using the synthetic sensor data as an input to a trained machine learning model, a predicted outcome for the patient; and output the predicted outcome.

In Example 59, the subject matter of Example 58 includes, wherein the model maps the time series of sensor data and the time series of wearable data based on timestamps of respective data points of each time series occurring within a threshold time range of each other.

In Example 60, the subject matter of Examples 58-59 includes, wherein the predicted outcome for the patient is determined without using any generated sensor data as an input.

In Example 61, the subject matter of Examples 58-60 includes, wherein the time series of sensor data includes at least one of accelerometer or gyroscope data, and wherein the time series of wearable data includes gait data.

Example 62 is at least one machine-readable medium including instructions, which when executed by processing circuitry, cause the processing circuitry to perform operations to: receive a patient-specific goal related to recovery from an orthopedic procedure on a patient; convert the patient-specific goal to a set of one or more metrics; receive sensor data generated by a sensor of a sensor device embedded in an orthopedic implant in the patient; determine, using a trained machine learning model and the sensor data, whether a metric of the set of one or more metrics is satisfied; in response to determining that all metrics of the set of one or more metrics are satisfied, output an indication that the patient-specific goal has been achieved; and in response to determining that the metric of the set of one or more metrics is not satisfied, output an indication corresponding to the metric.

In Example 63, the subject matter of Example 62 includes, wherein the indication corresponding to the metric includes an identified exercise to improve the metric.

In Example 64, the subject matter of Examples 62-63 includes, wherein the indication corresponding to the metric includes a predicted date when the patient-specific goal will be achieved.

In Example 65, the subject matter of Examples 62-64 includes, wherein the one or more metrics includes a range of motion for a joint.

In Example 66, the subject matter of Examples 62-65 includes, wherein the trained machine learning model is trained using historical sensor data with metric completion labels.

In Example 67, the subject matter of Examples 62-66 includes, wherein the indication that the patient-specific goal has been achieved includes information corresponding to a second patient-specific goal.

In Example 68, the subject matter of Examples 62-67 includes, wherein the patient-specific goal includes an identification of a life activity, and wherein the set of one or more metrics includes a range of motion corresponding to the life activity.

In Example 69, the subject matter of Examples 62-68 includes, wherein the patient-specific goal is received via a selection by the patient of the patient-specific goal on a user interface.

In Example 70, the subject matter of Examples 62-69 includes, wherein the operations further cause the processing circuitry to send, to the sensor device, an indication to activate one of a plurality of applications stored in memory of the sensor device, the plurality of applications preconfigured to be validated under a regulatory system, and wherein the received sensor data is generated according to the activated application.

In Example 71, the subject matter of Example 70 includes, wherein at least one operation of the sensor is changed when the activated application is activated.

Example 72 is a system comprising: a sensor device including a sensor to generate sensor data, the sensor device embedded in an orthopedic implant in a patient; a computing device including: processing circuitry; and memory including instructions, which when executed by the processing circuitry, cause the processing circuitry to perform operations to: receive a patient-specific goal related to recovery from an orthopedic procedure on the patient; convert the patient-specific goal to a set of one or more metrics; determine, using a trained machine learning model and the sensor data, whether a metric of the set of one or more metrics is satisfied; in response to determining that all metrics of the set of one or more metrics are satisfied, output an indication that the patient-specific goal has been achieved; and in response to determining that the metric of the set of one or more metrics is not satisfied, output an indication corresponding to the metric.

In Example 73, the subject matter of Example 72 includes, wherein the indication corresponding to the metric includes an identified exercise to improve the metric.

In Example 74, the subject matter of Examples 72-73 includes, wherein the indication corresponding to the metric includes a predicted date when the patient-specific goal will be achieved.

In Example 75, the subject matter of Examples 72-74 includes, wherein the one or more metrics includes a range of motion for a joint.

In Example 76, the subject matter of Examples 72-75 includes, wherein the trained machine learning model is trained using historical sensor data with metric completion labels.

In Example 77, the subject matter of Examples 72-76 includes, wherein the indication that the patient-specific goal has been achieved includes information corresponding to a second patient-specific goal.

In Example 78, the subject matter of Examples 72-77 includes, wherein the patient-specific goal includes an identification of a life activity, and wherein the set of one or more metrics includes a range of motion corresponding to the life activity.

In Example 79, the subject matter of Examples 72-78 includes, wherein the patient-specific goal is received via a selection by the patient of the patient-specific goal on a user interface.

In Example 80, the subject matter of Examples 72-79 includes, wherein the operations further cause the processing circuitry to send, to the sensor device, an indication to activate one of a plurality of applications stored in memory of the sensor device, the plurality of applications preconfigured to be validated under a regulatory system, and wherein the received sensor data is generated according to the activated application.

In Example 81, the subject matter of Example 80 includes, wherein at least one operation of the sensor is changed when the activated application is activated.

Example 82 is at least one machine-readable medium including instructions that, when executed by processing circuitry, cause the processing circuitry to perform operations to implement of any of Examples 1-81.

Example 83 is an apparatus comprising means to implement of any of Examples 1-81.

Example 84 is a system to implement of any of Examples 1-81.

Example 85 is a method to implement of any of Examples 1-81.

In some examples, any one or more components or operations described in any one or more of the Examples 1-81 may be included in any combination.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

1. A mobile device comprising:
processing circuitry; and
memory including instructions, which when executed by the processing circuitry, cause the processing circuitry to perform operations to:
receive, at a mobile device, compiled data generated by a sensor of a sensor device embedded in an orthopedic implant in a patient;
determine, based on patient-specific information, whether to use a local machine learning model operable at the mobile device, or a remote machine learning model operable at a remote device to output a prediction generated using the compiled data;
in accordance with a determination that the local machine learning model is to be used, predict, at the mobile device, an outcome for the patient by using the compiled data as an input to the local machine learning model;
in accordance with a determination that the remote machine learning model is to be used, send, from the mobile device, the compiled data to a remote computing device to generate a predicted outcome at the remote computing device using the compiled data.

2. The mobile device of claim 1, wherein the compiled data includes data pre-processed by the sensor device.

3. The mobile device of claim 1, wherein to determine whether to use the local machine learning model or the remote machine learning model includes operations to determine a current time frame related to an orthopedic procedure performed on the patient, and comparing the current time frame to a threshold time frame.

4. The mobile device of claim 1, wherein to determine whether to use the local machine learning model or the remote machine learning model includes operations to determine which model to use based on identifying an orthopedic procedure previously done on the patient.

5. The mobile device of claim 1, wherein to determine whether to use the local machine learning model or the remote machine learning model includes operations to determine which model to use based on an input received at the mobile device of at least one of a pain level identified by the patient, a range of motion of the patient, or a patient exercise score.

6. The mobile device of claim 1, wherein to determine whether to use the local machine learning model or the remote machine learning model includes operations to determine patient progress towards a goal defined by the patient.

7. The mobile device of claim 1, wherein to send the compiled data includes operations to sanitize the compiled data before being sent to remove personally identifying information from the compiled data.

8. The mobile device of claim 1, wherein a prediction by the local machine learning model is obtained in less time than a prediction by the remote machine learning model.

9. At least one machine-readable medium, including instructions for operation at a mobile device, which when executed, cause processing circuitry to perform operations to:
receive, at a mobile device, compiled data generated by a sensor of a sensor device embedded in an orthopedic implant in a patient;
determine, based on patient-specific information, whether to use a local machine learning model operable at the mobile device, or a remote machine learning model operable at a remote device to output a prediction generated using the compiled data;
in accordance with a determination that the local machine learning model is to be used, predict, at the mobile device, an outcome for the patient by using the compiled data as an input to the local machine learning model;
in accordance with a determination that the remote machine learning model is to be used, send, from the mobile device, the compiled data to a remote computing device to generate a predicted outcome at the remote computing device using the compiled data.

10. The at least one machine-readable medium of claim 9, wherein the compiled data includes data pre-processed by the sensor device.

11. The at least one machine-readable medium of claim 9, wherein to determine whether to use the local machine learning model or the remote machine learning model includes operations to determine a current time frame related to an orthopedic procedure performed on the patient, and compare the current time frame to a threshold time frame.

12. The at least one machine-readable medium of claim 9, wherein to determine whether to use the local machine learning model or the remote machine learning model includes operations to determine which model to use based on identifying an orthopedic procedure previously done on the patient.

13. The at least one machine-readable medium of claim 9, wherein to determine whether to use the local machine learning model or the remote machine learning model includes operations to determine which model to use based on an input received at the mobile device of at least one of a pain level identified by the patient, a range of motion of the patient, or a patient exercise score.

14. The at least one machine-readable medium of claim 9, wherein to determine whether to use the local machine learning model or the remote machine learning model includes operations to determine patient progress towards a goal defined by the patient.

15. The at least one machine-readable medium of claim 9, wherein to send the compiled data includes operations to sanitize the compiled data before being sent to remove personally identifying information from the compiled data.

16. The at least one machine-readable medium of claim 9, wherein a prediction by the local machine learning model is obtained in less time than a prediction by the remote machine learning model.

17. A system comprising:
a sensor device embedded in an orthopedic implant in a patient, the sensor device including:
a sensor to generate data; and
processing circuitry to compile the data; and
a mobile device including:
communication circuitry;
processing circuitry; and
memory including instructions, which when executed by the processing circuitry, cause the processing circuitry to perform operations to:
determine, based on patient-specific information, whether to use a local machine learning model operable at the mobile device, or a remote machine learning model operable at a remote device to output a prediction generated using the compiled data;
in accordance with a determination that the local machine learning model is to be used, predict, at the mobile device, an outcome for the patient by using the compiled data as an input to the local machine learning model;
in accordance with a determination that the remote machine learning model is to be used, send, from the communication circuitry of the mobile device, the compiled data to a remote computing device to generate a predicted outcome at the remote computing device using the compiled data.

18. The system of claim 17, wherein the compiled data includes data pre-processed by the processing circuitry within the sensor device.

19. The system of claim 17, wherein to determine whether to use the local machine learning model or the remote machine learning model includes operations to use at least one of a current time frame related to an orthopedic procedure performed on the patient, identification of an orthopedic procedure previously done on the patient, or an input received at the mobile device including at least one of a pain level identified by the patient, a range of motion of the patient, or a patient exercise score.

20. The system of claim 17, wherein to send the compiled data includes operations to sanitize the compiled data before being sent to remove personally identifying information from the compiled data.

21.-60. (canceled)
